SEQUENCE LISTING (9 sequences)

SEQ ID NO: 1 - protein, 395 aa, Homo sapiens
MPGRSCVALVLLAAAVSCAVAQHAPPWTEDCRKSTYPPSGPTYRGAVPWYTINLDLPPYKRWHELMLDKAPMLKVIVNSLKNMINTFVPSGKVMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDAMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 2 - protein, 394 aa, Mus musculus
MRGQSLLTWVLAAAVTCAQAQDVPPWTEDCRKSTYPPSGPTYRGPVPWHTINLDLPPYKRWHELLAQKAPALRILVNSITSLVNTFVPSGKLMKMVDQKLPGMIGSLPDPFGEEMRGIADVTGIPLGEIISFNIFYELFTMCTSIITEDEKGHLLHGRNMDFGIFLGWNINNNTWVVTEELKPLTVNLDFQRNNKTVFKATSFVGYVGMLTGFKPGLFSLSLNERFSINGGYLGILEWMFGRKDAQWVGFITRSVLENTTSYEEAKNTLTKTKIMAPVYFILGGKKSGEGCVITRERKESLDVYELDPKHGRWYVVQTNYDRWKNTLFIDDRRTPAKKCLNHTTQKNLSFATIYDVLSTKPVLNKLTVFTTLMDVTKGQFESHLRDCPDPCIGW

SEQ ID NO: 3 - protein, 395 aa, Gallus gallus
MAGRGRALLPPAVLMVLVLLVVAPDPYGEDCRSKMYPPSGPTFKGNVPTYIINLDLPPSKRWDELIRAKKTELKAVIQNIKDIANTFFPSGKIVDIVDHKISHLTDTLPYPFNEELQGIANSSGIPLGEIVIFNIFYEIFTVCTSIVAEDSRGKLYHARNLDFGLFLGWDVKNNFWTVTRELKPTVVNLDFQRNNKTVFRSTNFAGYIGMVSGVKPDLFTLTMNERFSLDGGYIGIFEWFLGRRDGMWMGFLTRSVLENATSYQDAKDKLAKTRLLAPAYFILGGKNSGEGCVITRSRTAALDIWDLDIKKGTWYVIETNYDRWKPPLVLDNRRTPAMKCLNQTSQENISLPTIYDVLSTKPVLNKLTVCTTLMEVDKGHTETYLRECPDPCSPW

SEQ ID NO: 4 - protein, 395 aa, Pan troglodytes
MPGRSRVALVLLAAAVSCAVAQHAPPWTEDCRKSTYPPSGPTYRGPVPWYTINLDLPPYKRWHELMLDKAPMLKVIVNSLKNMINTFVPSGKIVQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLSLNERFSINGGYLGILEWILGKKDAMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 5 - protein, 393 aa, Caenorhabditis elegans
MLRELSVLLLVAVCAAKHVELPAPFKDHCILDDKQNLYDPSKQFDIKWYDVNLDLPPSERWVQIATANKEHIADLIGVLINLITPWFPNAIDFVDDVFGDLAPKLAQPYRDEIFSIANATGIPLGQITMYNIFYEIFTVCTSVIAQDKDGHVFHARNLDFGLFMGWDPVLHDWQISQKLRKMIINVNWLKDGKLLYKSNNFAGYIGIYNGLKPNAFSLTADDRFQLVGGYYGILKWVFGLEADGKWMSWLARETLETKTTYLDAKEHLMNTPMLSPVYFILGGSKKDEGCIIARSLDKTALLTEMATSPHGWYLLETNYDQGTEDLYLDDRDTPGFRCMDKLTQKNVGFEGIFNVLSSRTNLNKLTTYTVLMSVETSRFETILQSCPGECYPW

SEQ ID NO: 6 - protein, 390 aa, Danio rerio
MKLVFRYNALFISIFIHALYVQGLEDCRSGMYPPKGPTYRGNVTRYTVNLDLPPSERWTQIIKDKNTELIEMVQTIKDMAKGFFHGKLVNFVDKELPFIVDTLPNPFNEEIKGIAAVSGIPLGEIALFNIFYEVFTVCTSLVAEDNNGNIYHGRNLDFGLFMGWDRQNKTWTLTEKLKPLVVNINFERKNQTVFKSTSFAGYVGMLTGIRPGELTLTMNERFDFDGGYIGILDWIFGNRDGMWTGFLTRRVLENSTSYEDAKDQLSQTKLLAPVYFILGGNRTGQGCVITRTRINTLDIWELELMLGRWYVLETNYDHWDKPMFLDDRRTPAMKCMNQTTQANISLASIYNVLSTKPVLNKLTTYTSLMAVSTGTLESYVRDCPNPCTPW

SEQ ID NO: 7 - protein, 394 aa, Rattus norvegicus
MLGRSLLTWVLAAAVTCAQAQQVPPWTEDCRKSTYPPSGPTYRGPVPWYTINLDLPPYKRWHELLAHKAPVLRTLVNSISNLVNAFVPSGKIMQMVDEKLPGLIGSIPGPFGEEMRGIADVTGIPLGEIISFNIFYELFTMCTSIITEDGKGHLLHGRNMDFGIFLGWNINNNTWVVTEELKPLTVNLDFQRNNKTVFKATSFAGYVGMLTGFKPGLLSLTLNERFSLNGGYLGILEWMFGKKNAQWVGFITRSVLENSTSYEEAKNILTKTKITAPAYFILGGNQSGEGCVITRERKESLDVYELDPKHGRWYVVQTNYDRWKNTLFLDDRRTPAKKCLNHTTQKNLSFATIYDVLSTKPVLNKLTVFTTLIDVTKDQFESHLRDCPDPCIGW

SEQ ID NO: 8 - protein, 395 aa, Homo sapiens
MPGRSCVALVLLAAAVSCAVAQHAPPWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 9 - protein, 395 aa, Homo sapiens; identical to SEQ ID NO: 8

(19) United States
(12) Patent Application Publication
(10) Pub. No.: US 2025/0144188 A1
(43) Pub. Date: May 8, 2025

(54) THERAPEUTIC ACID CERAMIDASE COMPOSITIONS AND METHODS OF MAKING AND USING THEM

(71) Applicant: ICAHN SCHOOL OF MEDICINE AT MOUNT SINAI, New York, NY (US)

(72) Inventor: Edward H. SCHUCHMAN, Haworth, NJ (US)

(21) Appl. No.: 18/750,693

(22) Filed: Jun. 21, 2024

Related U.S. Application Data
(63) Continuation of application No. 18/490,004, filed on Oct. 19, 2023, now abandoned.
Application No. 18/490,004 is a continuation of application No. 18/052,696, filed on Nov. 4, 2022, now abandoned.
Application No. 18/052,696 is a continuation of application No. 17/709,622, filed on Mar. 31, 2022, now abandoned.
Application No. 17/709,622 is a continuation of application No. 17/408,222, filed on Aug. 20, 2021, now abandoned.
Application No. 17/408,222 is a continuation of application No. 17/138,265, filed on Dec. 30, 2020, now abandoned.
Application No. 17/138,265 is a continuation of application No. 16/271,433, filed on Feb. 8, 2019, now Pat. No. 10,918,702.
Application No. 16/271,433 is a continuation of application No. 15/909,609, filed on Mar. 1, 2018, now Pat. No. 10,238,721.
Application No. 15/909,609 is a continuation of application No. 14/776,442, filed on Sep. 14, 2015, now Pat. No. 9,937,246.
(60) Provisional application No. 61/784,594, filed on Mar. 14, 2013.

(51) Int. Cl. A61K 38/50; A61K 38/43; A61K 38/46; A61K 45/06; C12N 9/80

(52) U.S. Cl. CPC A61K 38/50; A61K 38/43; A61K 38/46; A61K 45/06; C12N 9/80; C12Y 305/01023

(57) ABSTRACT

The present invention relates to a therapeutic composition including a ceramidase mixture and a pharmaceutically acceptable carrier, where the ceramidase mixture includes an inactive acid ceramidase precursor and an active acid ceramidase. The invention also relates to a method of acid ceramidase treatment, including formulating the acid ceramidase used in said treatment as a ceramidase mixture, where the ceramidase mixture includes an inactive acid ceramidase precursor and an active acid ceramidase. The invention further relates to a method of producing a therapeutic composition including providing a medium containing an inactive acid ceramidase precursor; incubating the medium under conditions effective to transform a portion of the inactive acid ceramidase precursor to active acid ceramidase; and recovering the incubated medium as a ceramidase mixture comprising the inactive acid ceramidase precursor and an active acid ceramidase. The present invention also relates to preparation of a therapeutic composition of a ceramidase lacking acid sphingomyelinase.

This application is a continuation of U.S. patent application Ser. No. 18/490,004, filed Oct. 19, 2023, which is a continuation of U.S. patent application Ser. No. 18/052,696, filed Nov. 4, 2022, which is a continuation of U.S. patent application Ser. No. 17/709,622, filed Mar. 31, 2022, which is a continuation of U.S. patent application Ser. No. 17/408,222, filed Aug. 20, 2021, which is a continuation of U.S. patent application Ser. No. 17/138,265, filed Dec. 30, 2020, which is a continuation of U.S. patent application Ser. No. 16/271,433, filed Feb. 8, 2019, which issued as U.S. Pat. No. 10,918,702 on Feb. 16, 2021, which is a continuation of U.S. patent application Ser. No. 15/909,609, filed Mar. 1, 2018, which issued as U.S. Pat. No. 10,238,721 on Mar. 26, 2019, which is a continuation of U.S. patent application Ser. No. 14/776,442, filed Mar. 13, 2014, which issued as U.S. Pat. No. 9,937,246 on Apr. 10, 2018, which is a national stage application under 35 U.S.C. § 371 of International Patent Application No. PCT/US2014/026481, filed Mar. 13, 2014, which claims the benefit of U.S. Provisional Patent Application Ser. No. 61/784,594, filed Mar. 14, 2013, which are hereby incorporated by reference in their entirety.

### SEQUENCE LISTING

The Sequence Listing is being submitted electronically in XML format and is hereby incorporated by reference in its entirety. Said XML copy, created on Jun. 20, 2024, is named 147535.000988.xml and is 12,098 bytes in Size. No new matter is being introduced.

### FIELD OF THE INVENTION

This invention relates to therapeutic acid ceramidase compositions and methods of making and using them.

### BACKGROUND OF THE INVENTION

Due to its involvement in the human genetic disorder Farber Lipogranulomatosis (“FD”), Acid ceramidase (“AC;” N-acylsphingosine deacylase, I.U.B.M.B. Enzyme No. EC 3.5.1.23) is the most extensively studied member of the ceramidase enzyme family. The protein has been purified from several sources, and the human and mouse cDNAs and genes have been obtained (Bernardo et al., “Purification, Characterization, and Biosynthesis of Human Acid Ceramidase,” J. Biol. Chem. 270:11098-102 (1995); Koch et al., “Molecular Cloning and Characterization of a Full-length Complementary DNA Encoding Human Acid Ceramidase. Identification of the First Molecular Lesion Causing Farber Disease,” J. Biol. Chem. 2711:33110-5 (1996); Li et al., “Cloning and Characterization of the Full-length cDNA and Genomic Sequences Encoding Murine Acid Ceramidase,” Genomics 50:267-74 (1998); Li et al., “The Human Acid Ceramidase Gene (ASAH): Chromosomal Location, Mutation Analysis, and Expression,” Genomics 62:223-31 (1999)). Growing interest in the biology of this and other ceramidases stems from the fact that these enzymes play a central role in ceramide metabolism.

Ceramide is a signaling lipid that is produced in response to various stimuli and extrinsic factors, including serum deprivation and treatment with many chemotherapy drugs, as well as in many human diseases (Hannun, “Function of Ceramide in Coordinating Cellular Responses to Stress,” Science 274:1855-9 (1996); Spiegel et al., “Signal Transduction Through Lipid Second Messengers,” Curr. Opin. Cell. Biol. 8:159-67 (1996)). Inside cells, ceramide can influence growth and differentiation, regulate protein secretion, induce DNA fragmentation and apoptosis, and increase the synthesis and secretion of cytokines. Normally present in low amounts, in response to these factors, ceramide is rapidly produced at the cell surface, leading to membrane re-organization and downstream signaling that results in apoptosis. After stimulation, AC and/or other ceramidases may then hydrolyze ceramide into the individual fatty acid and sphingosine components (Gatt, “Enzymic Hydrolysis and Synthesis of Ceramide,” J. Biol. Chem. 238:3131-3 (1963); Gatt, “Enzymatic Hydrolysis of Sphingolipids. 1. Hydrolysis and Synthesis of Ceramides by an Enzyme from Rat Brain,” J. Biol. Chem. 241:3724-31 (1966); Hassler & Bell, “Ceramidase: Enzymology and Metabolic Roles,” Adv. Lip. Res. 26:49-57 (1993)). Because ceramide degradation is the only source of intracellular sphingosine (Rother et al., “Biosynthesis of Sphingolipids: Dihydroceramide and Not Sphinganine Is Desaturated by Cultured Cells,” Biochem. Biophys. Res. Commun. 189:14-20 (1992)), these enzymes may also be rate-limiting steps in determining the intracellular levels of this compound. Importantly, a derivative of sphingosine, sphingosine-1-phosphate (“S1P”), can counteract the apoptotic effects of ceramide (Cuvillier et al., “Suppression of Ceramide-mediated Programmed Cell Death by Sphingosine-1-phosphate,” Nature 381:800-3 (1996)), leading to the suggestion that ceramidases can be “rheostats” that maintain a proper balance between cell growth and death (Spiegel & Merrill, “Sphingolipids Metabolism and Cell Growth Regulation,” FASEB J. 10:1388-97 (1996)).

AC hydrolyzes the amide bond linking the sphingosine and fatty acid moieties of the lipid ceramide (Park and Schuchman, “Acid Ceramidase and Human Disease,” Biochim. Biophys. Acta. 1758(12): 2133-2138 (2006)). Ceramide, sphingosine (and its phosphorylated derivative S1P) are bioactive lipids, and thus the activity of AC must be carefully regulated in cells (Young et al., “Sphingolipids: Regulators of Crosstalk Between Apoptosis and Autophagy,” J. Lipid Res. 54:5-19 (2013). One important mechanism by which AC activity is regulated is the cleavage of the inactive precursor polypeptide into the active enzyme consisting of an alpha and beta subunit linked via disulfide bonds (Shtraizent et al., “Autoproteolytic Cleavage and Activation of Human Acid Ceramidase,” J. Biol. Chem. 283:11253-11259 (2008)). It has previously been shown that recombinant AC produced in Chinese Hamster ovary (“CHO”) cells and secreted into the media is a mixture of inactive precursor and active (cleaved) enzyme (He et al., “Purification and Characterization of Recombinant, Human Acid Ceramidase,” J. Biol. Chem. 278:32978-32986 (2003)).

The present invention is directed to overcoming these and other deficiencies in the art.

### SUMMARY OF THE INVENTION

A first aspect of the present invention relates to a therapeutic composition including a ceramidase mixture and a pharmaceutically acceptable carrier. The ceramidase mixture includes an inactive acid ceramidase precursor and an active acid ceramidase.

A second aspect of the present invention relates to a method of acid ceramidase treatment, including formulating the acid ceramidase used in said treatment as a ceramidase mixture, where the ceramidase mixture includes an inactive acid ceramidase precursor and an active acid ceramidase.

A third aspect of the present invention relates to a method of producing a therapeutic composition. The method includes providing a medium containing an inactive acid ceramidase precursor and incubating the medium under conditions effective to transform a portion of the inactive acid ceramidase precursor to active acid ceramidase. The incubated medium is recovered as a ceramidase mixture comprising the inactive acid ceramidase precursor and an active acid ceramidase.

The present invention describes an optimal composition of recombinant AC (rAC). The present invention further describes the novel finding that, contrary to expectation, the fully active form of the enzyme is not the best form for promoting cell survival. Rather, preparations of purified rAC with higher amounts of inactive acid ceramidase (AC) precursor versus processed active AC are more effective at promoting cell survival and/or improving cell phenotype. Two preparations of recombinant AC were obtained containing different ratios of precursor and active enzyme. They were then used to evaluate the effects on the survival of oocytes in culture. Contrary to expectation, the preparation containing a higher ratio of inactive precursor had a greater effect on cell survival. It is hypothesized that this is due to the fact that the fully active enzyme has a shorter half-life in cells and in cell culture media. The same two preparations were tested using cultured primary chondrocytes. As with the oocytes, the preparation of recombinant AC with less of the active form had a greater effect on the expression of collagen 2, a marker of chondrogenesis.

rAC is being used experimentally in a number of cell systems and animal models to slow ceramide-related cell death and/or improve the phenotype of cells used for cell transplantation. It is also being studied in several disease models. The present invention describes the optimal preparation of rAC to be used for these purposes, which has numerous potential practical implications (e.g., in vitro fertilization, cartilage repair, and cystic fibrosis treatment).

In another derivative of the present invention, a novel method for the purification of recombinant AC was developed. In this method heat inactivation was used to remove acid sphingomyelinase and other contaminating proteins from the recombinant AC preparations. Previous work has shown that acid sphingomyelinase, a related lipid hydrolase, tightly binds to AC and co-purifies with it (Bernardo et al., “Purification, Characterization, and Biosynthesis of Human Acid Ceramidase,” J. Biol. Chem. 270:11098-11102 (1995), which is hereby incorporated by reference in its entirety). It has now been found that unlike most proteins, AC activity is fully stable when heated at 60° C. Thus, after column chromatography heat inactivation can be used to remove acid sphingomyelinase activity from the recombinant AC preparation.

Together, these two novel findings regarding (i) the importance of maintaining an optimal ratio of precursor and active AC, and (ii) the use of heat inactivation to remove acid sphingomyelinase activity and other contaminating proteins from the preparation, constitute unique and important observations regarding the composition of recombinant AC.

### BRIEF DESCRIPTION OF THE DRAWINGS

FIGS. 1A-1C show that preparations of rAC with less active ceramidase perform better than those with more active ceramidase. FIG. 1A is a western blot analysis showing the relative amounts of active (alpha/beta) versus inactive precursor rAC in two different bioreactor runs (Lot 6 and Lot 7). FIG. 1B summarizes results showing the ability of Lots 6 and 7 to form healthy mouse embryos. FIG. 1C depicts results of Lot 6 and Lot 7 after testing using cultured rat chondrocytes. At two weeks, the amount of collagen 2 expression was analyzed using western blotting.

FIG. 2 illustrates a time-response curve of AC and acid sphingomyelinase activity in Lot 7. Acid sphingomyelinase activity was removed without affecting AC activity.

FIG. 3 is a plot of acid ceramidase activity in (nmol/ml/hour) versus incubation time (in days).

FIG. 4 is a Western blot showing conversion of inactive acid ceramidase to active acid ceramidase.

FIGS. 5A-B are a Western blot showing conversion of inactive acid ceramidase to active acid ceramidase.

### DETAILED DESCRIPTION OF THE INVENTION

A first aspect of the present invention relates to a therapeutic composition including a ceramidase mixture and a pharmaceutically acceptable carrier. The ceramidase mixture includes an inactive AC precursor and an active AC.

Ceramidases hydrolyze the amide linkage of ceramides to generate free fatty acids and sphingoid bases (Nikolova-Karakashian et al., “Ceramidases,” Methods Enzymol. 311:194-201 (2000); Hassler et al., “Ceramidases: Enzymology and Metabolic Roles,” Adv. Lipid Res. 26:49-57 (1993), which are hereby incorporated by reference in their entirety). There are three types of ceramidases described to date (Nikolova-Karakashian et al., “Ceramidases,” Methods Enzymol. 311:194-201 (2000), which is hereby incorporated by reference in its entirety). These are classified as acid, neutral, and alkaline ceramidases according to their pH optimum of enzymatic activity.

ACs have optimal enzymatic activity at a pH<5.
The human AC was the first ceramidase to be cloned (Koch et al., “Molecular Cloning and Characterization of a Full-Length Complementary DNA Encoding Human Acid Ceramidase. Identification Of the First Molecular Lesion Causing Farber Disease,” J. Biol. Chem. 271:33110-33115 (1996), which is hereby incorporated by reference in its entirety). It is localized in the lysosome and is mainly responsible for the catabolismof ceramide. Dysfunction of this enzyme because of a genetic defect leads to a sphingolipidosis disease called Lipogranulomatosis or Farber disease (Koch et al., “Molecular Cloning and Characterization of a Full-Length Complementary DNA Encoding Human Acid Ceramidase. Identification Of the First Molecular Lesion Causing Farber Disease,” J. Biol. Chem. 271:33110-33115 (1996), Young et al., “Sphingolipids: Regulators of Crosstalk Between Apoptosis and Autophagy,” J. Lipid. Res. 54:5-19 (2013), which is hereby incorporated by reference in its entirety).

Inactive AC precursors and active ACs suitable for use in the ceramidase mixtures of this and all aspects of the present invention can be homologous (i.e., derived from the same species) or heterologous (i.e., derived from a different species) to the tissue, cells, and/or subject being treated. Ceramidase (e.g., AC) precursor proteins undergo autoproteolytic cleavage into the active form (composed of α- and β-subunits). The mechanism of human AC cleavage and activation is reported in Shtraizent et al., “Autoproteolytic Cleavage and Activation of Human Acid Ceramidase,” J. Biol. Chem. 283(17):11253-11259 (2008), which is hereby incorporated by reference in its entirety). This is promoted by the intracellular environment, and, based on highly conserved sequences at the cleavage site of ceramidase precursor proteins across species, is expected to occur in most, if not all, cell types. Thus, ceramidase as used herein includes both active ceramidases and ceramidase precursor proteins, where ceramidase precursor proteins are converted into active ceramidase proteins through autoproteolytic cleavage. Embodiments in which the precursor protein is taken up by the cell of interest and converted into active ceramidase thereby, as well as embodiments in which the precursor protein is converted into active ceramidase by a different cell or agent (present, for example, in a culture medium), are both contemplated.

AC (N-acylsphingosine deacylase, I.U.B.M.B. Enzyme No. EC 3.5.1.23) protein has been purified from several sources, and the human and mouse cDNAs and genes have been obtained. See Bernardo et al., “Purification, Characterization, and Biosynthesis of Human Acid Ceramidase,” J. Biol. Chem. 270:11098-102 (1995); Koch et al., “Molecular Cloning and Characterization of a Full-length Complementary DNA Encoding Human Acid Ceramidase. Identification of the First Molecular Lesion Causing Farber Disease,” J. Biol. Chem. 2711:33110-5 (1996); Li et al., “Cloning and Characterization of the Full-length cDNA and Genomic Sequences Encoding Murine Acid Ceramidase,” Genomics 50:267-74 (1998); Li et al., “The Human Acid Ceramidase Gene (ASAH): Chromosomal Location, Mutation Analysis, and Expression,” Genomics 62:223-31 (1999), all of which are hereby incorporated by reference in their entirety. It is produced through cleavage of the AC precursor protein (see Ferlinz et al., “Human Acid Ceramidase: Processing, Glycosylation, and Lysosomal Targeting,” J. Biol. Chem. 276(38):35352-60 (2001), which is hereby incorporated by reference in its entirety), which is the product of the AsahI gene (NCBI UniGene GeneID No. 427, which is hereby incorporated by reference in its entirety). AC protein [Homo sapien] (Accession No. AAC50907) is shown below in SEQ ID NO: 1.

| (SEQ ID NO: 1) | |
|---|---|
| 1 | mpgrscvalv llaaavscav aqhappwted crkstyppsg ptyrgavpwy tinldlppyk |
| 61 | rwhelmldka pmlkvivnsl knmintfvps gkvmqvvdek lpgllgnfpg pfeeemkgia |
| 121 | avtdiplgei isfnifyelf tictsivaed kkghlihgrn mdfgvflgwn inndtwvite |
| 181 | qlkpltvnld fqrnnktvfk assfagyvgm ltgfkpglfs ltlnerfsin ggylgilewi |
| 241 | lgkkdamwig fltrtvlens tsyeeaknll tktkilapay filggnqsge gcvitrdrke |
| 301 | sldvyeldak qgrwyvvqtn ydrwkhpffl ddrrtpakmc lnrtsgenis fetmydvlst |
| 361 | kpvlnkltvy ttlidvtkgq fetylrdcpd pcigw |

The AC alpha subunit begins at the amino acid at position 22 and continues through position 142 (as shown in bold in SEQ ID NO: 1), while the beta subunit of the AC begins with the amino acid at position 143 and continues through position 395 (as shown in italics in SEQ ID NO: 1).

Active ACs and inactive AC precursor proteins that can be used in this and all aspects of the present invention include, without limitation, those set forth in Table 1 below, which are hereby incorporated by reference in their entirety.

TABLE 1
Exemplary Acid Ceramidase Family Members

| | |
|---|---|
| Homo sapiens (SEQ ID NO: 1) | |
| UniProt | Q13510, Q9H715, Q96AS2 |
| OMIM | 228000 |
| NCBI Gene | 427 |
| NCBI RefSeq | NP_808592, NP_004306 |
| NCBI RefSeq | NM_177924, NM_004315 |
| NCBI UniGene | 427 |
| NCBI Accession | Q13510, AAC73009 |
| Mus musculus (SEQ ID NO: 2) | |
| UniProt | Q9WV54, Q3U8A7, Q78P93 |
| NCBI Gene | 11886 |
| NCBI RefSeq | NP_062708 |
| NCBI RefSeq | NM_019734 |
| NCBI UniGene | 11886 |
| NCBI Accession | AK151208, AK034204 |
| Gallus gallus (SEQ ID NO: 3) | |
| UniProt | Q5ZK58 |
| NCBI Gene | 422727 |
| NCBI RefSeq | NP_001006453 |
| NCBI RefSeq | NM_001006453 |
| NCBI UniGene | 422727 |
| NCBI Accession | CAG31885, AJ720226 |
| Pan troglodytes (SEQ ID NO: 4) | |
| NCBI Gene | 464022 |
| NCBI RefSeq | XP_519629 |
| NCBI RefSeq | XM_519629 |
| NCBI UniGene | 464022 |
| Caenorhabditis elegans (SEQ ID NO: 5) | |
| UniProt | O45686 |
| IntAct | O45686 |
| NCBI Gene | 173120 |
| NCBI RefSeq | NP_493173 |
| NCBI RefSeq | NM_060772 |
| NCBI UniGene | 173120 |
| NCBI Accession | O45686, CAB05556 |
| Danio rerio (SEQ ID NO: 6) | |
| UniProt | Q5XJR7 |
| NCBI Gene | 450068 |
| NCBI RefSeq | NP_001006088 |
| NCBI RefSeq | NM_001006088 |
| NCBI UniGene | 450068 |
| NCBI Accession | AAH83231, CB360968 |
| Rattus norvegicus (SEQ ID NO: 7) | |
| UniProt | Q6P7S1, Q9EQJ6 |
| NCBI Gene | 84431 |
| NCBI RefSeq | NP_445859 |
| NCBI RefSeq | NM_053407 |
| NCBI UniGene | 84431 |
| NCBI Accession | AAH61540, AF214647 |

| AC protein [Homo sapien] (Uniprot Q13510) is shown below in SEQ ID NO: 8. | | | | | | |
|---|---|---|---|---|---|---|
| (SEQ ID NO: 8) | | | | | | |
| MPGRSCVALV | LLAAAVSCAV | AQHAPPWTED | CRKSTYPPSG | PTYRGAVPWY | TINLDLPPYK | 61 |
| RWHELMLDKA | PVLKVIVNSL | KNMINTFVPS | GKIMQVVDEK | LPGLIGNEPG | PFEEEMKGIA | 121 |
| AVTDIPLGEI | ISENIFYELF | TICTSIVAED | KKGHLIHGRN | MDFGVELGWN | INNDTWVITE | 181 |
| QLKPLTVNLD | FORNNKTVFK | ASSFAGYVGM | LTGFKPGLFS | LTLNERFSIN | GGYLGILEWI | 241 |
| LGKKDVMWIG | FLTRTVLENS | TSYEEAKNLL | TKTKILAPAY | FILGGNOSGE | GCVITRDRKE | 301 |
| SLDVYELDAK | QGRWYVVQTN | YDRWKHPFFL | DDRRTPAKMC | LNRTSQENIS | FETMYDVIST | 361 |
| KPVINKLTVY | TTLIDVTKGQ | FETYLRDCPD | PCIGW | | | |
| AC protein [Homo sapien] (NCBI RefSeq NP_808592) is shown below in SEQ ID NO: 9. | | | | | | |
| (SEQ ID NO: 9) | | | | | | |
| MPGRSCVALV | LLAAAVSCAV | AQHAPPWTED | CRKSTYPPSG | PTYRGAVPWY | TINLDLPPYK | 61 |
| RWHELMLDKA | PVLKVIVNSL | KNMINTFVPS | GKIMQVVDEK | LPGLLGNFPG | PFEEEMKGIA | 121 |
| AVTDIPLGEI | ISENIFYELF | TICTSIVAED | KKGHLIHGRN | MDFGVELGWN | INNDTWVITE | 181 |
| QLKPLTVNLD | FQRNNKTVFK | ASSFAGYVGM | LTGFKPGLES | LTLNERFSIN | GGYLGILEWI | 241 |
| LGKKDVMWIG | FLTRTVLENS | TSYEEAKNLL | TKTKILAPAY | FILGGNOSGE | GCVITRDRKE | 301 |
| SLDVYELDAK | QGRWYVVQTN | YDRWKHPFEL | DDRRTPAKMC | LNRTSQENIS | FETMYDVLST | 361 |

| | | | |
|---|---|---|---|
| KPVLNKLTVY | TTLIDVTKGQ | FETYLRDCPD | PCIGW |

The ceramidase mixture of the therapeutic composition may, in some embodiments, contain a greater amount of the inactive AC precursor than active AC. Alternatively, the ceramidase mixture of the therapeutic composition may, in some instances, contain a lesser amount of inactive AC precursor than active AC.

In some embodiments, an effective amount of the inactive AC precursor compared to the active AC in the mixture ranges from about 5 to 95 wt % of the inactive AC precursor and 95 to 5 wt % of the active AC; 20 to 80 wt % of the inactive AC precursor and 80 to 20 wt % of the active AC; 30 to 70 wt % of the inactive AC precursor and 70 to 30 wt % of the active AC; 40 to 60 wt % of the inactive AC precursor and 60 to 40 wt % of the active AC; 55 to 95 wt % of the inactive AC precursor and 45 to 5 wt % of the active AC; 70 to 95 wt % of the inactive AC precursor and 30 to 5 wt % of the active AC; and may alternatively range from 80 to 90 wt % of the inactive AC precursor and 20 to 10 wt % of the active AC. An effective amount of the inactive AC precursor is 90 wt % while the active ceramidase is 10 wt % of the mixture. An alternative embodiment may include 80 wt % of the inactive ceramidase precursor and 20 wt % of the active AC in the ceramidase mixture. In yet a further embodiment, the ceramidase mixture may contain 60 wt % inactive ceramidase precursor and 40 wt % active ceramidase.

The therapeutic composition may also include pharmaceutically acceptable adjuvants, excipients, and/or stabilizers, and can be in solid or liquid form, such as tablets, capsules, powders, solutions, suspensions, or emulsions. Suitable adjuvants include, but are not limited to, flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles.

A second aspect of the present invention relates to a method of AC treatment, including formulating the AC used in said treatment as a ceramidase mixture, where the ceramidase mixture includes an inactive AC precursor and an active AC.

Treatment according to this aspect of the present invention is carried out using methods that will be apparent to the skilled artisan. For a discussion of AC in the context of human disease, see Park et al., “Acid Ceramidase and Human Disease,” Biochim. Phiophys. Act. 1758:2133-2138 (2006) and Zeidan et al., “Molecular Targeting of Acid Ceramidase: Implications to Cancer Therapy,” Curr. Drug Targets 9(8):653-661 (2008), both of which are hereby incorporated by reference in their entirety).

In some embodiments, treatment is carried out by introducing a ceramidase protein into the cells. An approach for delivery of proteins or polypeptide agents (e.g., active ceramidase, inactive ceramidase precursor proteins) involves the conjugation of the desired protein or polypeptide to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of proteins or polypeptide agents involves preparation of chimeric proteins according to U.S. Pat. No. 5,817,789 to Heartlein et al., which is hereby incorporated by reference in its entirety. The chimeric protein can include a ligand domain and the polypeptide agent (e.g., rAC, active AC, other ceramidase, inactive AC precursor protein, other ceramidase precursor proteins). The ligand domain is specific for receptors located on a target cell. Thus, when the chimeric protein is delivered to the cell, the chimeric protein will adsorb to the targeted cell, and the targeted cell will internalize the chimeric protein.

Further embodiments of the present aspect relate to methods of treatment for a certain disease or disorder. These methods involve formulating the AC used in the treatment as a ceramidase mixture including an inactive ceramidase precursor and an active AC.

In one embodiment, the disease or disorder is a joint disease or disorder and the ceramidase mixture according to the methods of the present invention is administered to a subject to treat the subject for the joint disease or disorder. Exemplary types of joint disease or disorders include, without limitation, osteoarthritis, rheumatoid arthritis, mucopolysaccharidosis, degenerative joint disease, joint injury, and Farber lipogranulomatosis.

In another embodiment, the disease or disorder is a neurodegenerative disease or disorder and the ceramidase mixture according to the methods of the present invention is administered to a subject to treat the subject for the neurodegenerative disease or disorder. Exemplary types of neurodegenerative diseases or disorders include, without limitation, Alzheimer's disease, Frontotemporal Dementia, Dementia with Lewy Bodies, Prion disease, Parkinson's disease, Huntington's disease, Progressive Supranuclear Palsy, Corticobasal Degeneration, Multiple System Atrophy, amyotrophic lateral sclerosis, inclusion body myositis, degenerative myopathy, spinocerebellar atrophy, metabolic neuropathy, diabetic neuropathy, endocrine neuropathy, orthostatic hypotension, brain injury, spinal cord injury, stroke, and motor neuron diseases such as spinal muscular atrophy.

In another embodiment, the disease or disorder is a cardiac disease or disorder and the ceramidase mixture according to the methods of the present invention is administered to a subject to treat the subject for the cardiac disease or disorder. Exemplary types of cardiac diseases or disorders include, without limitation, heart disease, cardiac injury, atherosclerosis, thrombosis, cardiomyocyte apoptosis, hypercardia, heart infarction, mitral regurgitation, aortic regurgitation, septal defect, and tachycardia-bradycardia syndrome.

In another embodiment, the disease or disorder is diabetes and the ceramidase mixture according to the methods of the present invention is administered to a subject to treat the subject for diabetes.

In another embodiment, the disease or disorder is a pathogenic infection in a subject having cystic fibrosis, chronic obstructive pulmonary disease (COPD), and/or an open wound, and the ceramidase mixture according to the methods of the present invention is administered to a subject to treat the subject for the pathogenic infection. Exemplary types of pathogenic infections include, without limitation, viral, fungal, prionic, and bacterial.

Subjects suffering from cystic fibrosis, COPD, and/or an open wound, may possess a high susceptibility for acquiring acute and/or chronic pathogenic infections, such as, e.g., bacterial, viral, fungal, protozoan, and/or prionic pathogenic infections. Bacterial pathogens include, without limitation, Bacillus anthracis, Bordetella pertussis, Borrelia burgdorferi, Campylobacter jejuni, Chlamydia trachomatis, Clostridium botulinum, Clostridium tetani, Corynebacterium dipththeriae, Escherichia coli, enterohemorrhagic E. coli, enterotoxigenic E. coli, Haemophilus influenzae type B and non-typable, Helicobacter pylori, Legionella pneumophila, Listeria monocytogenes, Mycobacterium spp., Mycobacterium leprae, Mycobacterium tuberculosis, Neisseria gonorrhoeae, Neisseria meningitidis, Pneumococcus spp., Pseudomonas aeruginosa, Rickettsia, Salmonella spp., Shigella spp., Staphylococcus spp., Staphylococcus aureus, Streptococcus spp., Streptococcus pneumoniae, Streptococcus pyogenes, Streptococcus B, Group A beta hemolytic Streptococcus, Streptococcus mutans, Treponema pallidum, Vibrio cholerae, and Yersinia pestis. In some embodiments, the pathogenic infection is a Pseudomonas infection.

Viral pathogens include, without limitation, RNA viruses, DNA viruses, adenovirdiae (e.g., mastadenovirus and aviadeno virus), herpesviridae (e.g., herpes simplex virus 1, herpes simplex virus 2, herpes simplex virus 5, and herpes simplex virus 6), leviviridae (e.g., levivirus, enterobacteria phage MS2, allolevirus), poxyiridae (e.g., chordopoxyirinae, parapoxvirus, avipoxvirus, capripoxvirus, leporipoxvirus, suipoxvirus, molluscipox virus, and entomopoxyirinae), papovaviridae (e.g., polyomavirus and papillomavirus), paramyxoviridae (e.g., paramyxovirus, parainfluenza virus 1, mobillivirus such as measles virus, rubulavirus (such as mumps virus), pneumonoviridae (e.g., pneumovirus, human respiratory syncytial virus), metapneumovirus (e.g., avian pneumovirus and human metapneumo virus), picornaviridae (e.g., enterovirus, rhinovirus, hepatovirus such as human hepatitis A virus, cardiovirus, and apthovirus), reoviridae (e.g., orthoreo virus, orbivirus, rotavirus, cypo virus, fijivirus, phytoreo virus, and oryzavirus), retroviridae (e.g., mammalian type B retroviruses, mammalian type C retroviruses, avian type C retroviruses, type D retrovirus group, BLV-HTLV retroviruses, lentivirus (such as human immunodeficiency virus 1 and human immunodeficiency virus 2; and spuma virus), flaviviridae (e.g., hepatitis C virus), hepadnaviridae (e.g., hepatitis B virus), togaviridae (e.g., alphavirus—such as sindbis virus and rubivirus, such as rubella virus), rhabdoviridae (e.g., vesiculovirus, lyssavirus, ephemera virus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus), Cytomegalovirus (mononucleosis), Dengue virus (dengue fever, shock syndrome), Epstein-Barr virus (mononucleosis, Burkitt's lymphoma), Human T-cell lymphotropic virus type 1 (T-cell leukemia), Influenza A, B, and C (respiratory disease), Japanese encephalitis virus (pneumonia, encephalopathy), Poliovirus (paralysis), Rhinovirus (common cold), Rubella virus (fetal malformations), Vaccinia virus (generalized infection), Yellow fever virus (jaundice, renal and hepatic failure), and Varicella zoster virus (chickenpox).

Pathogenic fungi include, without limitation, the genera Aspergillus (e.g., Aspergillus fumigates), Blastomyces, Candida (e.g., Candida albicans), Coccidiodes, Cryptococcus, Histoplasma, Phycomyces, Tinea corporis, Tinea unguis, Sporothrix schenckii, and Pneumocystis carinii. Pathogenic protozoan include, without limitation, Trypanosome spp., Leishmania spp., Plasmodium spp., Entamoeba spp., and Giardia spp. such as Giardia lamblia.

As described herein, an “open wound” refers to a type of injury in which an epithelial layer, i.e., skin, is torn, cut, and/or punctured. In some embodiments, an open wound refers to a sharp injury which damages the dermis of the skin and concomitantly increases the chance of acquiring an infection. The term “open wound” also encompasses burns.

In another embodiment, the disease or disorder is an infection caused by ceramide accumulation and the ceramidase mixture according to the methods of the present invention is administered to a subject to treat the subject for the ceramide accumulation infection.

The present invention may, in other embodiments, be used to treat Farber disease.

In at least one embodiment, treatment is carried out in vitro. In this embodiment, a ceramidase mixture can be taken from the subject or from a second subject then administered to the first subject (e.g., by injecting the mixture into the first subject). In at least one embodiment, treatment is carried out in vivo.

Mammalian subjects according to these aspects of the present invention include, for example, human subjects, equine subjects, porcine subjects, feline subjects, and canine subjects. Human subjects are particularly preferred.

In all embodiments that involve administering the ceramidase mixture to a subject, any combination of active ceramidase, ceramidase precursor protein, and/or nucleic acid encoding ceramidase/ceramidase precursor protein can be administered. Administration can be accomplished either via systemic administration to the subject or via targeted administration to affected tissues, organs, and/or cells. The ceramidase mixture may be administered to a non-targeted area along with one or more agents that facilitate migration of the ceramidase mixture to (and/or uptake by) a targeted tissue, organ, or cell. Additionally and/or alternatively, the ceramidase mixture itself can be modified to facilitate its transport to (and uptake by) the desired tissue, organ, or cell, as will be apparent to one of ordinary skill in the art.

Typically, the ceramidase mixture will be administered to a subject in a vehicle that delivers the ceramidase to the target cell, tissue, or organ. Exemplary routes of administration include, without limitation, orally, by inhalation, intratracheal inoculation, aspiration, airway instillation, aerosolization, nebulization, intranasal instillation, oral or nasogastric instillation, intraperitoneal injection, intravascular injection, topically, transdermally, parenterally, subcutaneously, intravenous injection, intra-arterial injection (such as via the pulmonary artery), intramuscular injection, intrapleural instillation, intraventricularly, intralesionally, intrathecally, by application to mucous membranes (such as that of the nose, throat, bronchial tubes, genitals, and/or anus), or implantation of a sustained release vehicle.

In some embodiments, the ceramidase mixture is administered orally, topically, intranasally, intraperitoneally, intravenously, subcutaneously, or by aerosol inhalation. In some embodiments, the ceramidase mixture is administered via aerosol inhalation. In some embodiments, the ceramidase mixture can be incorporated into pharmaceutical compositions suitable for administration, as described herein.

The ceramidase mixture may be orally administered, for example, with an inert diluent, or with an assimilable edible carrier, or they may be enclosed in hard or soft shell capsules, or they may be compressed into tablets, or may be incorporated directly with the food of the diet. For oral therapeutic administration, the ceramidase mixture may be incorporated with excipients and used in the form of tablets, capsules, elixirs, suspensions, syrups, and the like. Such compositions and preparations should contain at least 0.1% of ceramidase. The percentage of ceramidase mixture in these compositions may, of course, be varied and may conveniently be between about 2% to about 60% of the weight of the unit. The amount of the ceramidase mixture in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, or alginic acid; a lubricant such as magnesium stearate; and a sweetening agent such as sucrose, lactose, or saccharin. When the dosage unit form is a capsule, it may contain, in addition to materials of the above type, a liquid carrier, such as fatty oil.

The ceramidase mixture may also be administered parenterally. Solutions or suspensions of ceramidase can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The ceramidase mixture may also be administered directly to the airways in the form of an aerosol. For use as aerosols, ceramidase in solution or suspension may be packaged in a pressurized aerosol container together with suitable propellants, for example, hydrocarbon propellants like propane, butane, or isobutane with conventional adjuvants. The ceramidase mixture may also be administered in a non-pressurized form.

Exemplary delivery devices include, without limitation, nebulizers, atomizers, liposomes (including both active and passive drug delivery techniques) (Wang et al., “pH-Sensitive Immunoliposomes Mediate Target-cell-specific Delivery and Controlled Expression of a Foreign Gene in Mouse,” Proc. Nat'l Acad. Sci. USA 84:7851-5 (1987); Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al.; Wolff et al., “The Use of Monoclonal Anti-Thyl IgG1 for the Targeting of Liposomes to AKR-A Cells in Vitro and in Vivo,” Biochim. Biophys. Acta 802:259-73 (1984), each of which is hereby incorporated by reference in its entirety), transdermal patches, implants, implantable or injectable protein depot compositions, and syringes. Other delivery systems which are known to those of skill in the art can also be employed to achieve the desired delivery of ceramidase to the desired organ, tissue, or cells.

Administration can be carried out as frequently as required and for a duration that is suitable to provide effective treatment. For example, administration can be carried out with a single sustained-release dosage formulation or with multiple daily doses.

Treatment according to this and all aspects of the present invention may be carried out in vitro or in vivo. In vivo treatments include, for example, embodiments in which the population of cells is present in a mammalian subject. In such embodiments the population of cells can be either autologous (produced by the subject), homologous, or heterologous. Suitable subjects according to these embodiments include mammals, e.g., human subjects, equine subjects, porcine subjects, feline subjects, and canine subjects.

In one embodiment, one or more additional agents which reduce ceramide levels may be administered with the ceramidase mixture. This includes, without limitation, inhibitors of acid sphingomyelinase (e.g., amitryptiline (Becker et al., “Acid Sphingomyelinase Inhibitors Normalize Pulmonary Ceramide and Inflammation in Cystic Fibrosis,” Am. J. Respir. Cell. Mol. Biol. 42:716-724 (2010), which is hereby incorporated by reference in its entirety) and inhibitors of ceramide synthases (e.g., Shiffmann et al., “Inhibitors of Specific Ceramide Synthases,” Biochimie 94:558-565 (2012), which is hereby incorporated by reference in its entirety)).

The effective amount of a therapeutic agent/cell population of the present invention administered to the subject will depend on the type and severity of the disease or disorder and on the characteristics of the individual, such as general health, age, sex, body weight, and tolerance to drugs. It will also depend on the degree, severity, and type of disease or disorder. The skilled artisan will be able to determine appropriate dosages depending on these and other factors.

In one embodiment of the present invention, the method includes treating one or more mammalian cells ex vivo with said ceramidase mixture to promote cell survival. Cells whose survival can be promoted according to this aspect of the present invention include, without limitation, those that utilize the ceramidase apoptosis pathway, which includes a wide variety of cells (Obeid et al., “Programmed Cell Death Induced by Ceramide,” Science 259:1769-71 (1993), which is hereby incorporated by reference in its entirety), e.g., hepatocytes (Arora et al., “Ceramide Induces Hepatocyte Cell Death Through Disruption of Mitochondrial Function in the Rat,” Hepatol. 25:958-63 (1997), which is hereby incorporated by reference in its entirety), skin fibroblasts (Mizushima et al., “Ceramide, a Mediator of Interleukin 1, Tumour Necrosis Factor α, as Well as Fas Receptor Signalling, Induces Apoptosis of Rheumatoid Arthritis Synovial Cells,” Ann. Rheum. Dis. 57:495-9 (1998), which is hereby incorporated by reference in its entirety), chondrocytes (MacRae et al., “Ceramide Inhibition of Chondrocyte Proliferation and Bone Growth Is IGF-I Independent,” J. Endocrinol. 191(2):369-77 (2006), which is hereby incorporated by reference in its entirety), lung epithelium (Chan & Goldkorn, “Ceramide Path in Human Lung Cell Death,” Am. J. Respir. Cell Mol. Biol. 22(4):460-8 (2000), which is hereby incorporated by reference in its entirety), erythrocytes (Lang et al., “Mechanisms of Suicidal Erythrocyte Death,” Cell. Physiol. Biochem. 15:195-202 (2005), which is hereby incorporated by reference in its entirety), cardiomyocytes (Parra et al., “Changes in Mitochondrial Dynamics During Ceramide-induced Cardiomyocyte Early Apoptosis,” Cardiovasc. Res. (2007), which is hereby incorporated by reference in its entirety), and lymphocytes (Gombos et al., “Cholesterol and Sphingolipids as Lipid Organizers of the Immune Cells' Plasma Membrane: Their Impact on the Functions of MHC Molecules, Effector T-lymphocytes and T-cell Death,” Immunol. Lett. 104(1-2):59-69 (2006), which is hereby incorporated by reference in its entirety), eggs, embryos, neurons, sperm, synovial fibroblasts, and embryonic stem cells. Preferred cell types are eggs (fertilized or unfertilized), embryos, primary cells (e.g., neurons), sperm, synovial fibroblasts, and embryonic stem cells. Moreover, the ceramide apoptosis pathway appears to be conserved across mammalian species (Lee & Amoscato, “TRAIL and Ceramide,” Vitam. Horm. 67:229-55 (2004); see also, Samadi, “Ceramide-induced Cell Death in Lens Epithelial Cells,” Mol. Vis. 13:1618-26 (2007) (humans); Parra et al., “Changes in Mitochondrial Dynamics During Ceramide-induced Cardiomyocyte Early Apoptosis,” Cardiovasc. Res. (2007) (rat); de Castro E Paula et al., “Ceramide Inhibits Development and Cytokinesis and Induces Apoptosis in Preimplantation Bovine Embryos,” Mol. Reprod. Devel., DOI No. 10.1002/mrd.20841 (2007) (cows), each which is hereby incorporated by reference in its entirety). Therefore, it is expected that, for each of the cell types recited above, suitable cells include those of humans, monkeys, mice, rats, guinea pigs, cows, horses, sheep, pigs, dogs, and cats. This method may also be used to prolong the survival of eggs and/or embryos during in vitro fertilization procedures, facilitating the identification and selection of healthy embryos for reimplantation, especially for older human women and for veterinary breeding procedures.

Cells according to this aspect of the present invention can be provided by methods that will be apparent to the skilled artisan. By way of example, the cells can be obtained from an animal or from an existing ex vivo source (e.g., a tissue sample, a cell culture, etc.) using standard techniques. Treating cells ex vivo includes treating cells present in a homogeneous culture, as well as cells present in a heterogeneous culture (e.g., a tissue sample).

Inactive AC precursors and active ACs that can be used to prepare the ceramidase mixture in this and all aspects of the present invention include, without limitation, those set forth in Table 1, supra. In this and all aspects of the present invention (including the in vivo methods discussed below), the AC can be homologous (i.e., derived from the same species) or heterologous (i.e., derived from a different species) to the one or more cells being treated.

One embodiment of the present aspect of AC treatment relates to a method of producing chondrocytes with the ceramidase mixture. This method involves selecting a population of cells having the potential to differentiate into chondrocytes and treating the selected cell population with the ceramidase mixture to transform one or more of the cells in the selected population into chondrocytes.

Cells having the potential to differentiate into chondrocytes include bone marrow cells, fibroblasts, mesenchymal stem cells, and/or fibroblasts (see Mizushima et al., “Ceramide, a Mediator of Interleukin 1, Tumour Necrosis Factor a, as Well as Fas Receptor Signaling, Induces Apoptosis of Rheumatoid Arthritis Synovial Cells,” Ann. Rheum. Dis. 57:495-9 (1998), which is hereby incorporated by reference in its entirety).

Chondrocytes according to this aspect of the present invention include, without limitation, articular chondrocytes, nasal chondrocytes, tracheal chondrocytes, meniscal chondrocytes, and aural chondrocytes. These include, for example, mammalian chondrocytes, e.g., human chondrocytes, equine chondrocytes, porcine chondrocytes, feline chondrocytes, and canine chondrocytes. Preferably, the chondrocytes are primary chondrocytes.

Suitable cells according to this and all other aspects of the present invention include mammalian cells, e.g., human cells, equine cells, porcine cells, feline cells, and/or canine cells. Human cells are particularly preferred.

In this and all aspects of the present invention involving cell populations, embodiments in which the cells are all of one type, as well as embodiments in which the population is a mixture of two or more cell types, are both contemplated.

The ceramidase mixture and methods of treating the populations of cells with ceramidase mixture include all those set forth supra.

Another embodiment of the present aspect of AC treatment relates to a method of promoting chondrogenesis with the ceramidase mixture. In one embodiment, this method further includes selecting a population of stem cells in need of differentiation into chondrocytes, treating the population of stem cells with the ceramidase mixture to enrich mesenchymal stem cells within the stem cell population, and treating the population of enriched mesenchymal stem cells with the ceramidase mixture to promote differentiation of mesenchymal stem cells into chondrocytes.

Suitable cells populations according to this aspect of the present invention include mammalian cells populations, e.g., human cells populations, equine cells populations, porcine cells populations, feline cells populations, and/or canine cells populations. Human cells populations are particularly preferred.

Suitable stem cells according to this and all other aspects of the present invention include bone marrow cells, adipocytes, and skin cells. Additional stem cells according to this aspect of the present invention include, without limitation, embryonic stem cells, somatic stem cells, induced pluripotent stem cells, totipotent stem cells, pluripotent stem cells, and multipotent stem cells. Exemplary stem cells include, for example, hematopoietic stem cells, mesenchymal stem cells, neural stem cells, endothelial progenitor cells, epithelial stem cells, epidermal stem cells, adipocytes, and cardiac stem cells. Suitable stem cells include, but are not limited to, mammalian cells, e.g., human, equine, porcine, feline, and canine bone marrow cells, adipocytes, and skin cells. Human cells are particularly preferred.

Suitable chondrocytes are consistent with those described supra. The differentiated mesenchymal stem cells may, alternatively, be primary cells such as, but not limited to, neurons, hepatocytes, bone cells, lung cells, and cardiac cells.

In at least one embodiment, the number of differentiated cells in the cell population is maintained. In at least one embodiment, the number of differentiated cells in the cell population is increased. As will be apparent to the skilled artisan, maintaining or increasing the overall number of differentiated cells in the population can be achieved by decreasing or preventing de-differentiation of cells in the population that are already differentiated, by stimulating the differentiation of undifferentiated cells in the population, or both.

The ceramidase mixture and methods of treating the populations of cells with ceramidase mixture include all those set forth supra.

A third aspect of the present invention relates to a method of producing a therapeutic composition. The method includes providing a medium containing an inactive AC precursor; incubating the medium under conditions effective to transform a portion of the inactive AC precursor to active AC; and recovering the incubated medium as a ceramidase mixture comprising the inactive AC precursor and an active AC.

The therapeutic composition of the present invention contains a recombinant protein including both inactive AC precursor and active AC. The recombinant protein of the present invention may be prepared for use in the above described methods of the present invention using standard methods of synthesis known in the art, including solid phase peptide synthesis (Fmoc or Boc strategies) or solution phase peptide synthesis. Alternatively, proteins of the present invention may be prepared using recombinant expression systems.

Generally, the use of recombinant expression systems involves inserting the nucleic acid molecule encoding the amino acid sequence of the desired peptide into an expression system to which the molecule is heterologous (i.e., not normally present). One or more desired nucleic acid molecules encoding a peptide of the invention may be inserted into the vector. When multiple nucleic acid molecules are inserted, the multiple nucleic acid molecules may encode the same or different peptides. The heterologous nucleic acid molecule is inserted into the expression system or vector in proper sense (5′→3′) orientation relative to the promoter and any other 5′ regulatory molecules, and correct reading frame.

The preparation of the nucleic acid constructs can be carried out using standard cloning procedures well known in the art as described by Joseph Sambrook et al., MOLECULAR CLONING: A LABORATORY MANUAL (Cold Springs Harbor 1989). U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation into a suitable host cell.

A variety of genetic signals and processing events that control many levels of gene expression (e.g., DNA transcription and messenger RNA (“mRNA”) translation) can be incorporated into the nucleic acid construct to maximize peptide production. For the purposes of expressing a cloned nucleic acid sequence encoding a desired recombinant protein, it is advantageous to use strong promoters to obtain a high level of transcription. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR. Mammalian cells that may be used for manufacture of the recombinant protein of the present invention include, for example, Chinese Hamster Ovary (CHO) cells, plant cells, chicken eggs, and human fibroblasts.

There are other specific initiation signals required for efficient gene transcription and translation in prokaryotic cells that can be included in the nucleic acid construct to maximize peptide production. Depending on the vector system and host utilized, any number of suitable transcription and/or translation elements, including constitutive, inducible, and repressible promoters, as well as minimal 5′ promoter elements, enhancers or leader sequences may be used. For a review on maximizing gene expression see Roberts and Lauer, “Maximizing Gene Expression On a Plasmid Using Recombination In Vitro,” Methods in Enzymology 68:473-82 (1979), which is hereby incorporated by reference in its entirety.

A nucleic acid molecule encoding a recombinant protein of the present invention, a promoter molecule of choice, including, without limitation, enhancers, and leader sequences; a suitable 3′ regulatory region to allow transcription in the host, and any additional desired components, such as reporter or marker genes, are cloned into the vector of choice using standard cloning procedures in the art, such as described in Joseph Sambrook et al., MOLECULAR CLONING: A LABORATORY MANUAL (Cold Springs Harbor 1989); Frederick M. Ausubel, SHORT PROTOCOLS IN MOLECULAR BIOLOGY (Wiley 1999), and U.S. Pat. No. 4,237,224 to Cohen and Boyer, which are hereby incorporated by reference in their entirety.

Once the nucleic acid molecule encoding the peptide has been cloned into an expression vector, it is ready to be incorporated into a host. Recombinant molecules can be introduced into cells, without limitation, via transfection (if the host is a eukaryote), transduction, conjugation, mobilization, or electroporation, lipofection, protoplast fusion, mobilization, or particle bombardment, using standard cloning procedures known in the art, as described by JOSEPH SAMBROOK et al., MOLECULAR CLONING: A LABORATORY MANUAL (Cold Springs Harbor 1989), which is hereby incorporated by reference in its entirety.

A variety of suitable host-vector systems may be utilized to express the recombinant protein or polypeptide. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria.

Purified peptides may be obtained by several methods readily known in the art, including ion exchange chromatography, hydrophobic interaction chromatography, affinity chromatography, gel filtration, and reverse phase chromatography. The peptide is preferably produced in purified form (preferably at least about 80% or 85% pure, more preferably at least about 90% or 95% pure) by conventional techniques. Depending on whether the recombinant host cell is made to secrete the peptide into growth medium (see U.S. Pat. No. 6,596,509 to Bauer et al., which is hereby incorporated by reference in its entirety), the peptide can be isolated and purified by centrifugation (to separate cellular components from supernatant containing the secreted peptide) followed by sequential ammonium sulfate precipitation of the supernatant. In one embodiment of the present invention, cells may be transformed with DNA encoding AC and then cultured under conditions effective to produce the medium containing inactive AC precursor. The fraction containing the peptide is subjected to gel filtration in an appropriately sized dextran or polyacrylamide column to separate the peptides from other proteins. If necessary, the peptide fraction may be further purified by other chromatography.

In one embodiment of the present invention, the incubation is carried out under conditions effective to reduce the transformation rate of inactive AC precursor to active AC compared to the transformation rate achieved when said incubating is carried out at a pH of 4 and a temperature of 4° C. or 37° C., for 24 hours, under otherwise consistent conditions. Alternatively, the incubating may be carried out under conditions effective to enhance the transformation rate of inactive AC precursor to active AC compared to those same conditions.

In some embodiments, the ceramidase mixture during the incubating may have a pH over 4.0 and up to 6.5. The mixture may, for example, have a pH of 4.0, 4.5, 5.0, 5.5, 6.0, or 6.5. In other embodiments, the temperature of the ceramidase mixture during said incubating may be at least −30° C. and under 37° C. The temperature of the mixture may, for example, be −30° C., −25° C., −20° C., −15° C., −10° C., −5° C., 0° C., 5° C., 10° C., 15° C., 20° C., 25° C., 30° C., or 35° C. Alternatively, the mixture may be incubated under conditions of −30° C. with a pH of 4.0, 4° C. with a pH of 4.0 or 6.5, 25° C. with a pH of 4.0, or 37° C. with a pH of 4.0. The mixture may be incubated for a period of time such as, but not limited to, approximately 30 minutes, 1 hour, 3 hours, 30 hours, or 300 hours.

During incubation of this aspect of the present invention, the medium may be heated under conditions effective to remove acid sphingomyelinase activity. In this embodiment, the medium may be heated to 60° C. for a period of time including, but not limited to, less than 20 minutes, 20-40 minutes, 40-60 minutes, or more than 60 minutes.

It is to be appreciated that certain aspects, modes, embodiments, variations and features of the present invention are described in various levels of detail in order to provide a substantial understanding of the present technology. The definitions of certain terms as used in this specification are also provided. Unless defined otherwise, all technical and scientific terms used herein generally have the same meaning as commonly understood by one of ordinary skill in the art to which this invention belongs.

### EXAMPLES

The following examples are provided to illustrate embodiments of the present invention but are by no means intended to limit its scope.

### Example 1—Materials and Methods

Preparation of rAC (Lot 6 and Lot 7)—Chinese hamster ovary cells overexpressing the human Asah1 gene were generated and rAC was purified from the media (He et al., “Purification and Characterization of Recombinant, Human Acid Ceramidase. Catalytic Reactions and Interactions With Acid Sphingomyelinase,” J. Biol. Chem. 278:32978-32986 (2003), which is hereby incorporated by reference in its entirety). No in vitro manipulation was carried out after purification of Lot 6 (higher amount of inactive AC precursor). After purification of Lot 7 enzyme, the rAC was incubated in pH 4 citrate phosphate buffer at 37° C. for three hours.

Comparison of Lot 6 and Lot 7 for Mouse Embryo Production—Methods for using rAC for mouse embryo production are described in Eliyahu et al., “Acid Ceramidase Improves the Quality of Oocytes and Embryos and the Outcome of In Vitro Fertilization,” FASEB J. 24:1229-1238 (2010), which is hereby incorporated by reference in its entirety. Sperm and mature oocytes were obtained from C57 Black mice and in vitro fertilization was carried out using equal amounts of Lot 6 and Lot 7 enzyme.

Comparison of Lot 6 and Lot 7 to Improve the Chondrogenic Phenotype of Rat Articular Chondrocytes—Equal amounts of Lot 6 and Lot 7 rAC were added to the media of primary articular chondrocytes isolated from femurs. Cartilage was digested and cells were placed into culture with and without rAC supplementation. After three days the media was changed to media without rAC. Cells were grown for an additional two weeks and the levels of collagen 2 (marker of mature articular chondrocytes) was determined by western blotting.

### Example 2—rAC With Less Active AC Perform Better Than Those With More Active AC

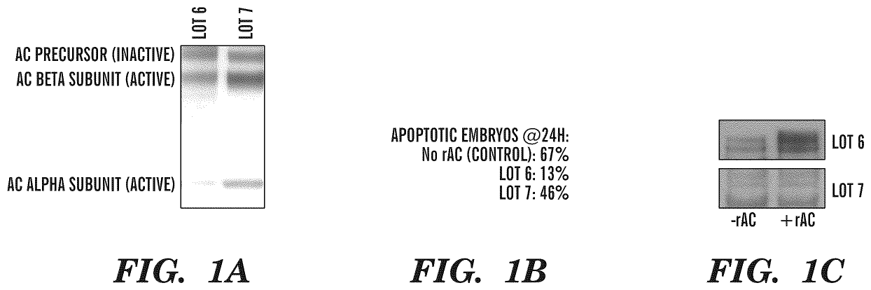

Preparations of rAC with less active ceramidase perform better than those with more active ceramidase (FIGS. 1A-1C). As indicated in FIG. 1A, a western blot analysis showed the relative amounts of active (alpha/beta) versus inactive precursor rAC in two different bioreactor runs (Lot 6 and Lot 7). Lot 7 had more active rAC than in Lot 6. In vitro examples (IVF and chondrocytes) compared two rAC preparations with ratios that were approximately 90:10 (inactive: active) (Lot 6) versus approximately 80:20 (inactive: active) (Lot 7) (FIG. 1A). Apoptosis was determined at 24 hours using standard morphological methods (e.g., membrane integrity, etc.) (Eliyahu et al., “Acid Ceramidase Improves the Quality of Oocytes and Embryos and the Outcome of In Vitro Fertilization,” FASEB J. 24:1229-1238 (2010), which is hereby incorporated by reference in its entirety).

FIG. 1B summarizes results for ability to form healthy mouse embryos in Lot 6 and Lot 7. Lot 7 (containing more active rAC) produced more apoptotic embryos than lot 6 (containing less active rAC). As can be seen in FIG. 1B, the preparation with less active enzyme (Lot 6) provided better results in IVF (fewer apoptotic embryos). This was unexpected.

Lot 6 and Lot 7 were also tested using cultured rat chondrocytes (FIG. 1C). At two weeks the amount of collagen 2 expression was analyzed using western blotting. Cells cultured with Lot 7 (more active rAC) produced less collagen 2. Lot 6 was better in maintaining the chondrocyte phenotype after expansion (FIG. 1C, based on the expression of collagen 2). This was unexpected.

The improved performance of rAC containing less active AC is hypothesized to be due to the shorter half-life of the active enzyme in cultured cells (conversely the longer half-life of the precursor).

In order to manipulate the ratio of inactive to active enzyme, pH was adjusted to 4.0 and the preparation was incubated at 37° C. Under these conditions an increase of approximately 10% active enzyme was observed for every 3 hours of incubation. Thus, to covert a preparation that is 90:10 inactive:active to 100% active, the preparation is incubated for 27 hours.

An important variable here is temperature. If the preparations are maintained (pH adjusted) frozen, there is no conversion. If the preparations are maintained at 4° C. (in a refrigerator) the conversion proceeds but at 1% the efficiency of 37° C. (10% increase in active enzyme requires 300 versus 3 hours). If the preparation is maintained at room temperature (25° C.), it proceeds at 10% the efficiency (10% increase requires 30 hours). If the pH is not acidified, there is no conversion at 4° C. and only 1% conversion rate (300 hours are required for an increase of 10%) at room temperature.

### Example 3—Removal of Contaminating Acid Sphingomyelinase Activity From rAC

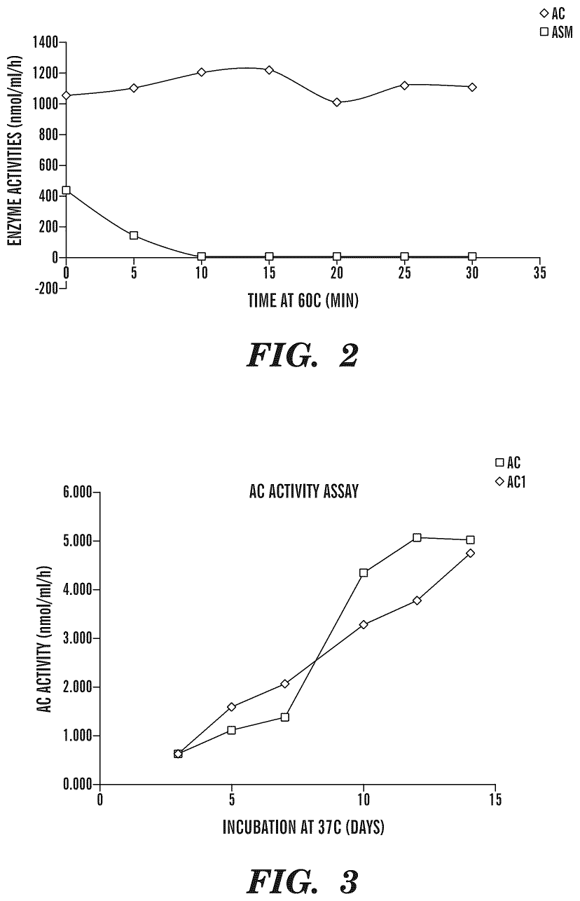

Methods of removing contaminating acid sphingomyelinase activity (ASM) from the rAC preparations were developed. This requires incubation of the final rAC preparations at 60° C. for 10-20 minutes. This incubation does not affect rAC (activity or ratio of inactive to active) but removes all ASM activity, which is essential to manufacturing (FIG. 2).

### Example 4—Incubation of Media Containing Recombinant Human Acid Ceramidase at 37° C. for Varying Lengths of Time

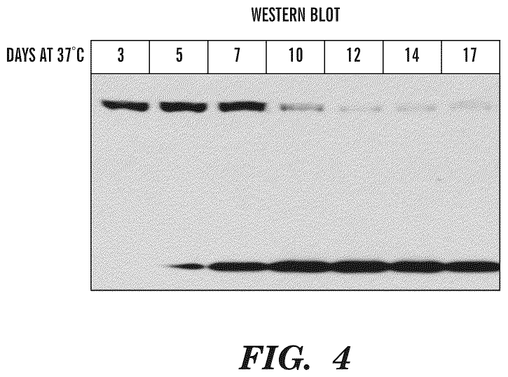

Conditioned media (DMEM, pH 6.8 containing 10% fetal calf serum) was collected from Chinese hamster ovary cells overexpressing and secreting recombinant human acid ceramidase (rhAC) (He et al., “Purification and Characterization of Recombinant, Human Acid Ceramidase. Catalytic Reactions and Interactions With Acid Sphingomyelinase,” J. Biol. Chem. 278:32978-32986 (2003), which is hereby incorporated by reference in its entirety). The cells were grown until ˜100% confluency in T-75 mm flasks, and media was then collected after 4 days of additional growth. The collected media was filtered through 0.22 mm membranes to removed debris and placed in a 37° C. incubator for varying lengths of time. At the end of the incubation period the media was frozen at −20° C. prior to assay. AC activity (FIG. 3) was determined as previously described (He et al., Anal Biochem, 274:264 (1999), which is hereby incorporated by reference in its entirety): reaction mixtures were incubated at 37° C. for one hour. AC Western Blot (FIG. 4): 6.5 μl/lane, was developed using a mouse anti-human AC monoclonal antibody (1:300, #SC136275, Santa Cruz) against the alpha-subunit. This data shows that in vitro incubation of media containing rhAC at 37° C. for 3-17 days, resulting in conversion of inactive precursor into active enzyme (represented by the alpha subunit and an increase in enzymatic activity).

### Example 5—In Vitro Conversion of Purified, Recombinant Human Acid Ceramidase at 37° C.

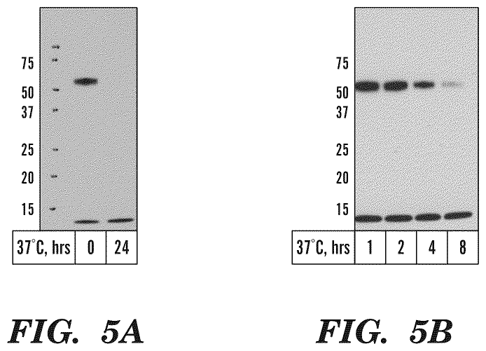

Purified recombinant human AC (rhAC; 4 ug/ul in EMEM, pH 6.8) was isolated from the media of overexpressing Chinese hamster ovary cells as previously described (He et al., “Purification and Characterization of Recombinant, Human Acid Ceramidase. Catalytic Reactions and Interactions With Acid Sphingomyelinase,” J. Biol. Chem. 278:32978-32986 (2003), which is hereby incorporated by reference in its entirety). AC Western Blot (FIG. 5): 6.5 μl/lane, was developed using a mouse anti-human AC monoclonal antibody against the alpha-subunit (1:300, #SC136275, Santa Cruz). This data shows that in vitro incubation of purified rhAC at 37° C. for 24 h (FIG. 5A) resulted in complete conversion of the precursor to active form. Incubation from 1-8 hours (FIG. 5B) showed a linear progression of conversion.

Although the invention has been described in detail for the purpose of illustration, it is understood that such detail is solely for that purpose, and variations can be made therein by those skilled in the art without departing from the spirit and scope of the invention which is defined by the following claims.

1. A method of producing a therapeutic composition, the method comprising:
providing a medium containing an inactive acid ceramidase precursor;
incubating the medium under conditions effective to transform a portion of the inactive acid ceramidase precursor to active acid ceramidase; and
recovering the incubated medium as a ceramidase mixture comprising the inactive acid ceramidase precursor and an active acid ceramidase.

2. The method of claim 1, wherein said incubating is carried out under conditions effective to reduce the transformation rate of inactive acid ceramidase precursor to active acid ceramidase compared to the transformation rate achieved when said incubating is carried out at a pH of 4 and a temperature of 4° C. or 37° C., for 24 hours, under otherwise consistent conditions.

3. The method of claim 2, wherein the pH of the ceramidase mixture during said incubating is over 4.0 and up to 6.5.

4. The method of claim 2, wherein the temperature of the ceramidase mixture during said incubating is at least −30° C. and under 37° C.

5. The method of claim 1, wherein said incubating comprises:
heating the medium under conditions effective to remove acid sphingomyelinase activity.

6. The method according to claim 1, wherein said providing a medium comprises:
providing cells transformed with DNA encoding acid ceramidase and
culturing the transformed cells under conditions effective to produce the medium containing inactive acid ceramidase precursor.

7. (canceled)

8. A therapeutic composition comprising:
a ceramidase mixture comprising:
an inactive acid ceramidase precursor and
an active acid ceramidase; and
a pharmaceutically acceptable carrier.

9. The therapeutic composition of claim 8, wherein, in the ceramidase mixture, the amount of the inactive acid ceramidase precursor is greater than the amount of the active acid ceramidase or, wherein, in the ceramidase mixture, the amount of the inactive acid ceramidase precursor is less than the amount of the active acid ceramidase.

10. (canceled)

11. The therapeutic composition of claim 8, wherein the ceramidase mixture comprises 5 to 95 wt % of the inactive acid ceramidase precursor and 95 to 5 wt % of the active acid ceramidase.

12. (canceled)

13. The therapeutic composition of claim 11, wherein the ceramidase mixture comprises 30 to 70 wt % of the inactive acid ceramidase precursor and 70 to 30 wt % of the active acid ceramidase, 40 to 60 wt % of the inactive acid ceramidase precursor and 60 to 40 wt % of the active acid ceramidase, 55 to 95 wt % of the inactive acid ceramidase precursor and 45 to 5 wt % of the active acid ceramidase, 70 to 95 wt % of the inactive acid ceramidase precursor and 30 to 5 wt % of the active acid ceramidase, 80 to 90 wt % of the inactive acid ceramidase precursor and 20 to 10 wt % of the active acid ceramidase, or 20 to 80 wt % of the inactive acid ceramidase precursor and 80 to 20 wt % of the active acid ceramidase.

14.-17. (canceled)

18. The therapeutic composition of claim 8 further comprising:
an adjuvant, wherein the adjuvant is selected from the group consisting of flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles.

19. (canceled)

20. In a method of acid ceramidase treatment, the improvement comprising:
formulating the acid ceramidase used in said treatment as a ceramidase mixture comprising:
an inactive acid ceramidase precursor and
an active acid ceramidase.

21.-24. (canceled)

25. The method of claim 20, wherein the method comprises:
promoting chondrogenesis with said ceramidase mixture.

26. The method of claim 25, wherein the method comprises:
selecting a population of stem cells in need of differentiation into chondrocytes;
treating the population of stem cells with said ceramidase mixture to enrich mesenchymal stem cells within the stem cell population; and
treating the population of enriched mesenchymal stem cells with said ceramidase mixture to promote differentiation of mesenchymal stem cells into chondrocytes.

27. The method of claim 26, wherein the mesenchymal stem cells are mammalian.

28. The method of claim 20, wherein the method comprises:
administering said ceramidase mixture to a subject to treat the subject for a joint disease or disorder.

29. The method of claim 28, wherein the joint disease or disorder is selected from the group consisting of osteoarthritis, rheumatoid arthritis, mucopolysaccharidosis, degenerative joint disease, joint injury, and Farber lipogranulomatosis.

30. The method of claim 20, wherein the method comprises:
administering said ceramidase mixture to a subject to treat the subject for a neurodegenerative disease or disorder.

31. The method of claim 30, wherein the neurodegenerative disease or disorder is selected from the group consisting of Alzheimer's disease, Frontotemporal Dementia, Dementia with Lewy Bodies, Prion disease, Parkinson's disease, Huntington's disease, Progressive Supranuclear Palsy, Corticobasal Degeneration, Multiple System Atrophy, amyotrophic lateral sclerosis, inclusion body myositis, degenerative myopathy, spinocerebellar atrophy, metabolic neuropathy, diabetic neuropathy, endocrine neuropathy, orthostatic hypotension, brain injury, spinal cord injury, stroke, and motor neuron diseases such as spinal muscular atrophy.

32.-43. (canceled)
